(19) United States
(12) Patent Application Publication
(10) Pub. No.: US 2011/0202012 A1
(43) Pub. Date: Aug. 18, 2011

(54) Smart Injection Syring Systems Providing Real-Time User Feedback of Correct Needle Position

(76) Inventor: Edwin C. Bartlett, Greenville, NC (US)

(21) Appl. No.: 12/672,958

(22) PCT Filed: Aug. 14, 2008
(86) PCT No.: PCT/US08/09722

§ 371 Date: Feb. 10, 2010

Related U.S. Application Data
(60) Provisional application No. 60/956,235, filed on Aug. 16, 2007.

(51) Int. Cl. A61M 5/31; G09B 23/28; A61M 5/315

(52) U.S. Cl. 604/218; 604/187; 434/262

(57) ABSTRACT

Syringe assemblies include a syringe with a syringe body (15b) defining a fluid cavity in fluid communication with an injection needle (25); a force, pressure and/or flow sensor (30) in fluid communication with the needle; and a user feedback unit (22) in electrical communication with the sensor and configured to provide user feedback based on data from the force, pressure and/or flow sensor.

### RELATED APPLICATIONS

This application claims priority to U.S. Provisional Patent Application No. 60/956,235, filed Aug. 16, 2007, the disclosure of which is hereby incorporated by reference in its entirety.

### FIELD OF THE INVENTION

The invention relates to syringes and may be particularly suitable for syringes that inject medicaments into joint spaces.

### BACKGROUND OF THE INVENTION

It is routine in the course of treating musculoskeletal complaints, injuries, and disease to utilize injections of several types for the relief of pain and inflammation or to promote cartilage repair. Injection types include steroid injection, local anesthetics, hyaluronic acid, or mixtures of the above. One aspect of performing this injection is the proper location of the injected fluid. Unfortunately, some health care providers perform numerous injections with limited knowledge and training in surface anatomy, tissue planes, and musculoskeletal compartments. This knowledge can be the difference in successful diagnosis, treatment, and often pain relief for the suffering patient. While a basic understanding of deep and surface anatomy is required for success, proper injection technique often affords the highest rate of success.

The ability to sense the nature of the space into which one is injecting can be important to successful location of the chosen fluid. An experienced operator can typically sense the nature of the tissue he or she is injecting into by interpretations of the excursion rate and fluid resistance he or she feels while pressing on the plunger of the syringe. For example, if the tip of the injecting needle is buried in the substance of a tendon, the operator will encounter high resistance and very limited flow of the fluid. If however, the needle tip is located in a joint space, the fluid will flow easily with limited resistance. This tactile ability is not particularly intuitive and is dependent on proper equipment as well as operator skill. For instance, many providers will choose to use the smallest bore needle available (on the assumption that it will cause less pain), but the resistance from a 25 gauge needle can be enough to negate one's ability to sense the nature of the tissues into which the fluid is directed. Switching to a larger needle, e.g., a 21 gauge needle, permits the desired sensing feedback to an experienced operator while causing minimally increased discomfort from using a slightly larger needle. However, the size of the syringe can cause variations in the tactile response/sensing of the injection.

Routinely, the operator is looking to inject into an anatomical cavity where the resistance is much less that an injection directly into a tissue. A device can assist the untrained, inexperienced, or tactilely-challenged operator in sensing this location may provide significant benefit to the patient.

### SUMMARY OF EMBODIMENTS OF THE INVENTION

Embodiments of the present invention are directed to syringes that can provide visual or audio location feedback to a user in substantially real-time to facilitate the proper site delivery of medicament to a desired location in the body.

Some embodiments are directed to syringe assemblies that include a syringe with a syringe body defining a fluid cavity in fluid communication with an injection needle; a force, pressure and/or flow sensor in fluid communication with the needle; and a user feedback unit in electrical communication with the sensor and configured to provide user feedback based on data from the force, pressure and/or flow sensor.

The user data feedback unit can include a housing having a light indicator whereby in operation the housing generates a green light if the needle is in a suitable location for injection. The housing can be configured to releasably engage the syringe body. The housing can be configured to slidably snugly receive a portion of the syringe therein. The user feedback unit can be attached to the syringe.

In some embodiments, the housing or syringe body can include a digital signal processor circuit configured to calculate an index of resistance associated with the location of the needle in a patient with a low index of resistance indicating a desired injection site. The sensor can be configured to wirelessly communicate the digital signal processor circuit.

Some embodiments are directed to orthopedic syringes used to treat musculoskeletal complaints, injuries, pain and/or disease to joints. Particular embodiments are directed to syringes used to inject anti-inflammatory agents, such as corticosteroids and/or hyaluronic acid, and/or to injectsubcutaneously into joint cavity spaces, such as knee joints, shoulder joints, elbow joints, finger joints and the like.

Embodiments of the invention provide syringes that can calculate a resistance index associated with an excursion flow rate into a subject and generate a visible confirmation that the syringe needle is in the correct target space by generating, for example, a “green” light.

Embodiments of the invention may comprise a shell or outer casing that communicates with a disposable syringe. The shell or outer casing can comprise at least one visual indicator light that can alert a user as to whether the syringe is in the proper location. The syringe can include a pressure or force sensor that wirelessly communicates with the shell or casing to generate the visual confirmation of correct location.

Some embodiments of the invention may comprise a syringe system including a syringe with a syringe body defining a fluid cavity and having a plunger in the fluid cavity. The fluid cavity can be in fluid communication with an injection needle. A plunger force sensor can be configured to detect a force exerted on the plunger by a user. A displacement sensor can be configured to detect a displacement of the plunger. A user feedback unit can be in communication with the plunger force sensor and the displacement sensor and can be configured to provide user feedback based on data from the force and/or flow sensor.

Some embodiments of the invention may comprise a syringe sensor system configured to releasably attach to a syringe with a syringe body defining a fluid cavity and having a plunger in the fluid cavity. The fluid cavity can be in fluid communication with an injection needle. The syringe sensor system can include at least one housing configured to releasably attach to the syringe. A plunger force sensor can be on the at least one housing and can be configured to detect a force exerted on the plunger by a user. A displacement sensor can be on the at least one housing and can be configured to detect a displacement of the plunger. A user feedback unit can be in electrical communication with the plunger force sensor and the displacement sensor and configured to provide user feedback based on data from the force and/or flow sensor.

Some embodiments of the invention may comprise methods for selecting a tissue region for injection with a syringe in communication with an injection needle. A force, pressure and/or flow associated with the syringe and/or injection needle can be detected during insertion of the needle and/or during injection of a fluid through the needle. User feedback can be provided responsive to the detected force, pressure and/or flow.

Embodiments of the invention provide syringes that can be easy to use, and even by patients, for chronic injections with disposable syringes that cooperate with a reusable ergonomic housing, shell or casing. Embodiments of the invention provide syringes that can be used as a teaching aid for nurses, doctors or other clinicians to “learn” the correct anatomical delivery space by teaching the user the tactile feel of the correct anatomical delivery space by associating the tactile feel with the digital confirmation of location based on the syringe's capacity to digitally monitor pressure and/or flow rate and/or calculate a resistance index, then provide substantially real-time feedback to a user.

It is noted that any of the features claimed with respect to one type of claim, such as a system, apparatus, or computer program, may be claimed or carried out as any of the other types of claimed operations or features.

Further features, advantages and details of the present invention will be appreciated by those of ordinary skill in the art from a reading of the figures and the detailed description of the embodiments that follow, such description being merely illustrative of the present invention.

### BRIEF DESCRIPTION OF THE DRAWINGS

FIG. 1 is a schematic illustration of a syringe with a “smart” readout or alert that confirms that the needle is in a proper anatomical space according to embodiments of the present invention.

FIGS. 2A and 2B are partial schematic illustrations of user feedback configurations that provide an indication of proper injection location of a needle in substantially real-time according to embodiments of the present invention.

FIGS. 3A-3C are schematic illustrations of syringe delivery systems with user feedback according to embodiments of the present invention.

FIG. 4 is a perspective view of a syringe system according to embodiments of the present invention.

FIG. 5 is a reusable plunger housing of the syringe system of FIG. 4.

FIG. 6 is a reusable syringe housing of the syringe system of FIG. 4.

FIG. 7 is a graph of the voltage as a function of force of a plunger force sensor on the syringe system of FIG. 4 for calibration according to embodiments of the present invention.

FIG. 8 is a graph of the displacement as a function of voltage for a displacement sensor on the syringe system of FIG. 4 for calibration according to embodiments of the present invention.

FIG. 9 is a graph of the force and displacement as a function of time for joint tissue according to embodiments of the present invention.

FIG. 10 is a graph of the force and displacement as a function of time for tendon tissue according to embodiments of the present invention.

FIG. 11 is a scatter plot of the pressure as a function of flow-rate for tendon and joint tissue as a function of time.

### DETAILED DESCRIPTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity. Broken lines illustrate optional features or operations unless specified otherwise.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention. The sequence of operations (or steps) is not limited to the order presented in the claims or figures unless specifically indicated otherwise.

The term “excursion” rate refers to the discharge or flow rate of the liquid medicament out of the syringe body and/or needle into local anatomical structure.

The term “electrical communication” as used herein includes both wired and wireless communication between elements.

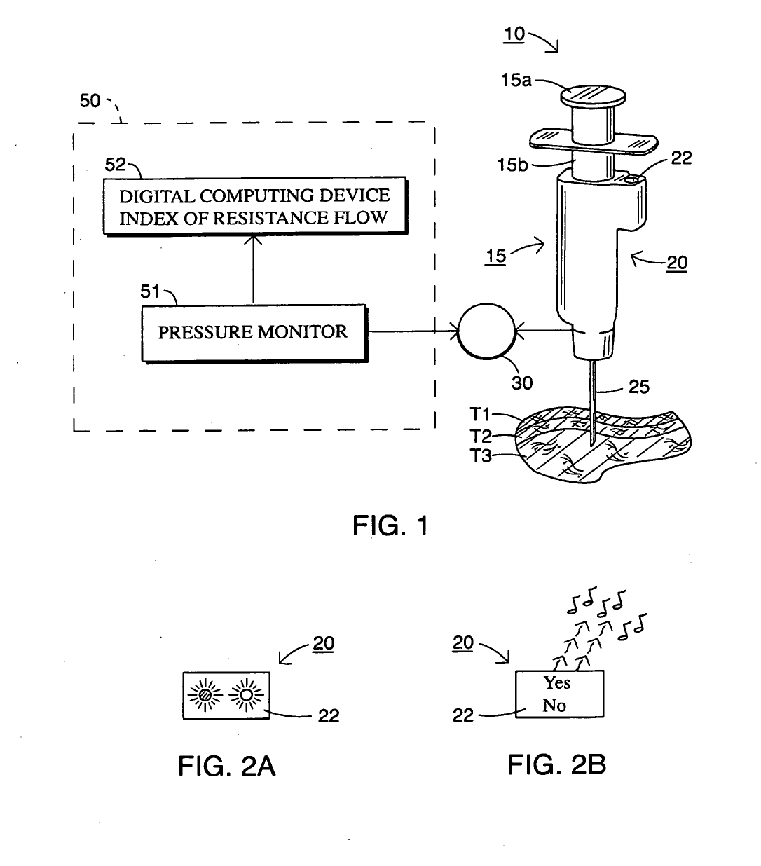

As shown in FIG. 1, the medical device 10 includes an injection syringe 15 with a plunger 15a and a syringe body 15b in communication with a user feedback unit 22. As shown in FIG. 1, the syringe body 15b is in fluid communication with a needle 25, and the needle 25 is in fluid communication with at least one sensor 30 such as a force, pressure and/or flow sensor. The device 10 further includes a syringe housing 20. The at least one sensor 30 can include both pressure (or force) sensor and a flow sensor (such as flow meter). The at least one sensor 30 can be held on the housing 20 and/or be incorporated into the syringe body 15b.

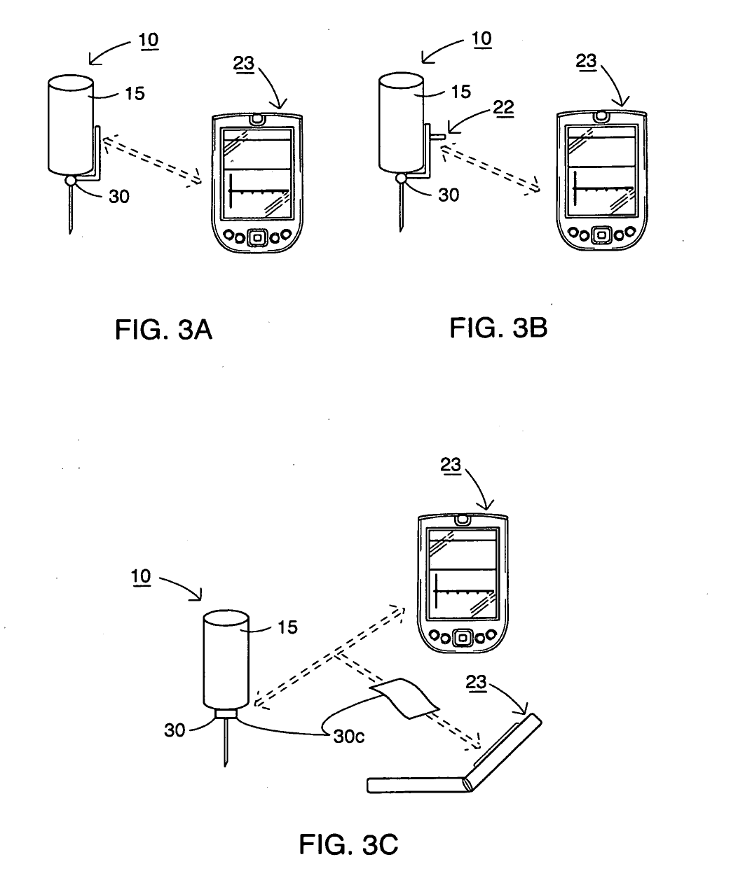

The sensor 30 can wirelessly communicate with a digital signal processing circuit 50 that can be incorporated into the housing 20 and/or a discrete local or remote computer device 23 (FIGS. 3A-3C) and, when used, the remote computer device 23 can wirelessly transmit the user feedback signal to the “onboard” user feedback unit 22.

In this configuration, when the needle 25 is inserted into different types of body tissue T1, T2, and T3, the output from the sensor(s) 30 can be used to provide user feedback via the user feedback unit 22 to indicate when the desired insertion location has been reached. For example, T1 may be fat tissue, T2 may be muscle or tendon tissue, and T3 may be a joint cavity, and the desired needle insertion location may be the joint cavity (T3).

The user feedback unit 22 can include a light indicator or display and can be held in a case (e.g., housing) 20 attached to the syringe body 15b as shown in FIG. 1. In some embodiments, as shown in FIG. 3A, the user feedback unit 22 may reside in or comprise a discrete remote or local pervasive computing device 23, such as a wireless communication device, such as, for example, a cellular wireless telephone or PDA, and/or a laptop or other computer that communicates with the at least one sensor 30. The device 10 can also be configured to include one or both of the on-board user feedback unit 22 and a display in the computer device 23. As shown in FIG. 3B, the device 10 can include a casing or housing 20 with a light emitter that provides the user feedback unit 22 with light and the user feedback unit 22 wirelessly communicates with the computer device 23 which directs the appropriate output by the user feedback unit 22.

Again, as shown in FIG. 1, the user feedback unit 22 is held by or attached to the housing 20. The housing 20 can be lightweight and ergonomic and can releasbly snugly slidably or frictionally serially engage different syringes 15. The housing 20 can be compact and cover only a portion of a syringe body 15b, thereby allowing a user visual contact with the syringe fluid in the cavity of the syringe body. The housing 20 can be multi-use while the syringes 15 can be single-use disposable. The housing 20 may optionally omit a user feedback indicator or member. In certain embodiments, as shown in FIG. 3C, the syringe 15 may be configured with the at least one sensor 30 integrated therein or thereon and can include a wireless transmission circuit 30c to communicate with at least one portable communications device 23 (shown as two) without requiring an additional housing component.

The housing 20 can be configured as a lightweight balanced device that does not provide eccentric weight or unbalance or unduly affect the injection operation.

The user feedback unit 22 can provide visual or audio feedback to a user in substantially real-time to confirm or alert a user as to the proper or improper position of the needle in situ. The user feedback unit 22 can be provided as part of the housing 20 or as a separate device. As shown in FIG. 2A, the user feedback unit 22 can comprise side-by-side LEDs (green and red or other suitable colors). In operation, a “green” LED light can be activated when appropriate to inject based on pressure and flow feedback and/or the index of resistance. Alternatively, a single LED could be used whereby no light would indicate either a “go” or “no go” position of the needle per instructions and training. As shown in FIG. 2B, other user feedback units 22 may be used such as, for example, a small integrated display that can generate visual and/or audio output to a user in substantially real-time, e.g., a musical note or tone for yes or no, or actual words such as “stop,” “yes,” “no,” “go,” “okay,” etc. may be output as either an audio output, as a visible readable output or both.

FIG. 1 also illustrates that the device 10 can include a digital processor circuit 50 that can be in electrical communication with the sensor 30. The digital processor circuit 50 can be held on the syringe integral with a sensor circuit 30 (e.g., be “onboard” with the syringe), but is typically held in the housing 20 and/or in a local or remote computer device 23 (FIGS. 3A-3C). The digital processor circuit 50 can wirelessly communicate with the sensor 30 to obtain the desired sensor data. The circuit 50 and/or the sensor 30 can include a pressure monitor 51. The circuit 50 can comprise computer readable program code configured to programmatically direct the user feedback and may calculate an index of flow resistance 52 whereby a low index of resistance value confirms that the needle 25 is in a proper anatomical location, such as a desired joint space. In contrast, a high index of resistance indicates an improper location (e.g., in a tendon rather than a joint space). Other measures of flow or resistance may be used.

Thus, in some embodiments, the syringe assembly 10 can comprise a computer device 23 that calculates and/or measures resistance and/or calculates an index of resistance and flow in order to alert a user as to location of the needle relative to a comparison with a desired range of these parameters for proper placement of an injected fluid. The syringe assembly 10 can have an outer shell or housing 20 to which disposable syringes and needles could be attached. A clearly visible user feedback indicator member 22 (e.g., red light, green light) on the housing 20 could then signal the conditions for fluid injection.

The wireless communication between the electronic components can be carried out using a BLUETOOTH transmission configuration or any other suitable digital communication protocol or configuration. Virtual reality position sensing can also be utilized depending on cost factors. The device 10 can be configured as an easy-to-use and economical medical tool to promote more reliable and accurate injection of medicament to a target space.

Any suitable sensor or combination of sensors can be used for the sensor 30. Embodiments according to the present invention will now be described with respect to the following non-limiting examples.

### Example 1

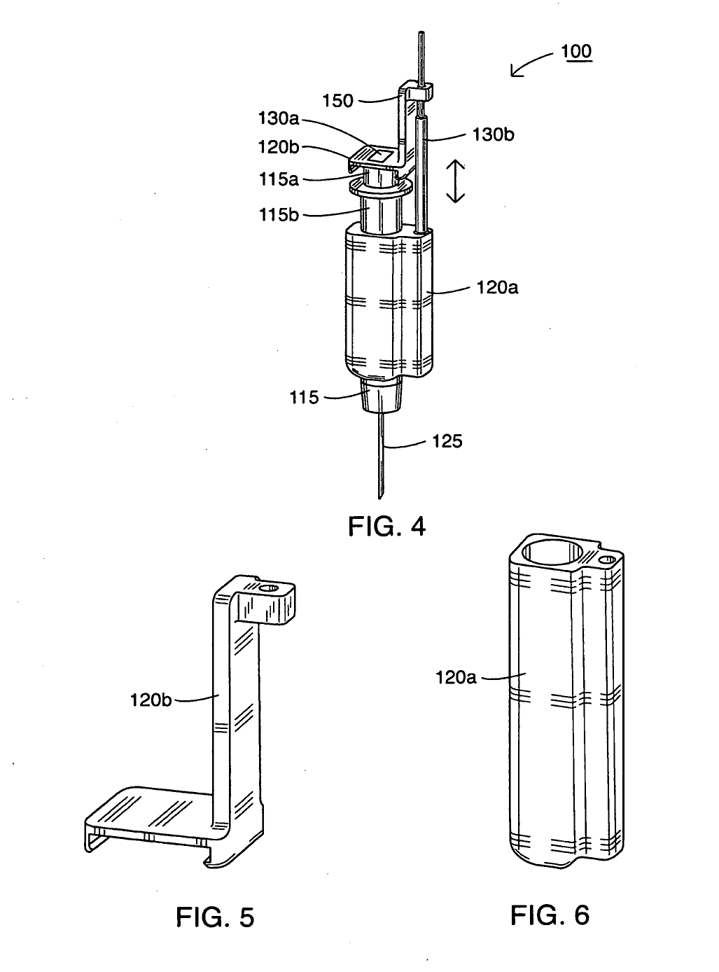

As illustrated in FIGS. 4-6, a syringe system 100 includes a syringe 115 with a syringe plunger 115a in a syringe body 115b, which is in communication with a needle 125. Two support members or housings 120a, 120b are configured to connect sensors 130a, 130b to the syringe 115. The sensor 130a is a force sensor configured to detect an amount of force with which the user depresses the plunger 115a. As illustrated in FIG. 4, the sensor 130a is connected to the plunger 115a by the housing 120b. The sensor 130b is a displacement sensor that is in communication with the plunger 115a and detects the displacement of the plunger 115a as the user depresses the plunger 115a.

In operation, the force sensor 130a detects the force that the user applies to the plunger 115a, and the displacement sensor 130b detects the displacement of the plunger 115a as the plunger 115a is depressed. Accordingly, the sensors 130a, 130b are positioned on the outside components of the syringe 115 such that the sensors 130a, 130b do not contact the fluid in the syringe 115, and modification to the syringe 115 may be reduced or eliminated.

The force information from the sensor 130a can be converted into fluid pressure information because the fluid pressure can be calculated as the force divided by the relevant area. The displacement information from the sensor 130b can be converted into flow information by calculating the volume of fluid displaced by the plunger 115a and dividing by the time during which the displacement occurs. The sensors 130a, 130b detect force and displacement information, for example, when the user makes a relatively small test injection. The pressure and flow readings from the sensors 130a, 130b can then be converted into tissue impedance, e.g., by the computer device 23 and relayed to the user via the user feedback indicator unit 22 as discussed with respect to FIGS. 1-3. If the user feedback indicator unit 22 indicates that the desired location has been reached based on the tissue impedance, then the user can continue with a full injection. If the desired location has not been reached, the user can maneuver the needle 125 to another location and deliver another test injection.

In particular embodiments, the force sensor 130a is a FlexiForce A201 sensor (Tekscan, Inc., South Boston, Mass., USA) that senses between 0-25 lbs of force with a sensing area diameter of 0.375 inches. Any suitable force sensor can be used, for example, sensors can be used that operate within a general dynamic force range for an injection (e.g., about 2-3 lbs. of force) and with a sensing area that is sufficiently small to be mounted to the top of the syringe plunger 115a.

In particular embodiments, the displacement sensor 130b is an S-VDRT, 38 mm range displacement transducer (MicroStrain, Williston, Vt., USA), which measures sufficient displacement of the plunger 115a while being sufficiently small to be mounted on the syringe 115.

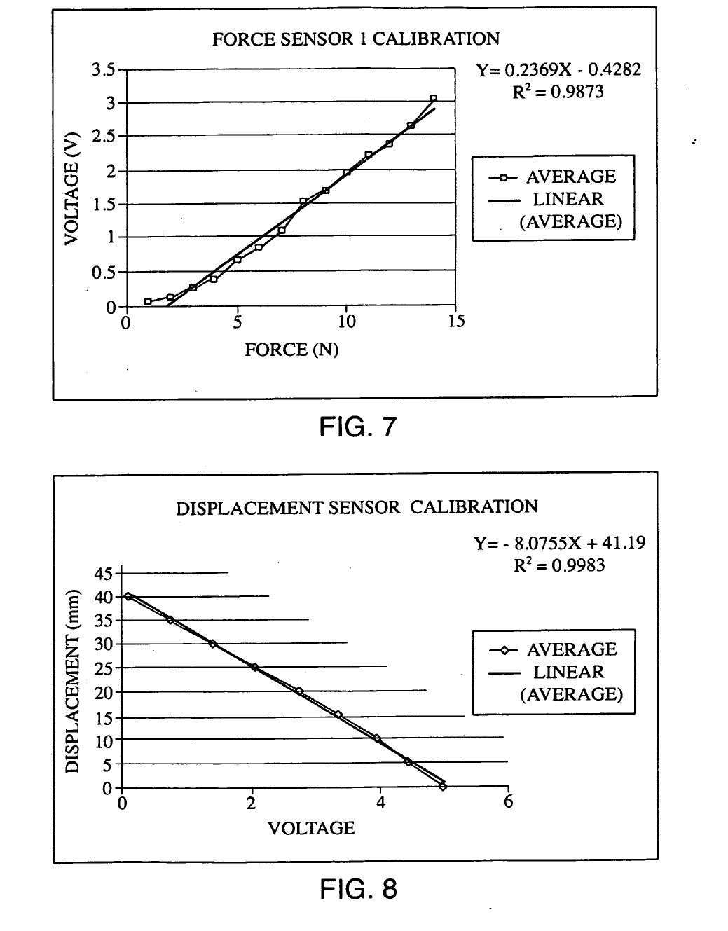

The sensors 130a, 130b can be attached to a circuit and/or software to manipulate data and/or provide a user feedback indicator member 22, e.g., to convert voltage readings from the sensors into units of force and displacement, respectively. Exemplary force and displacement sensor calibrations are illustrated in FIGS. 7 and 8, respectively.

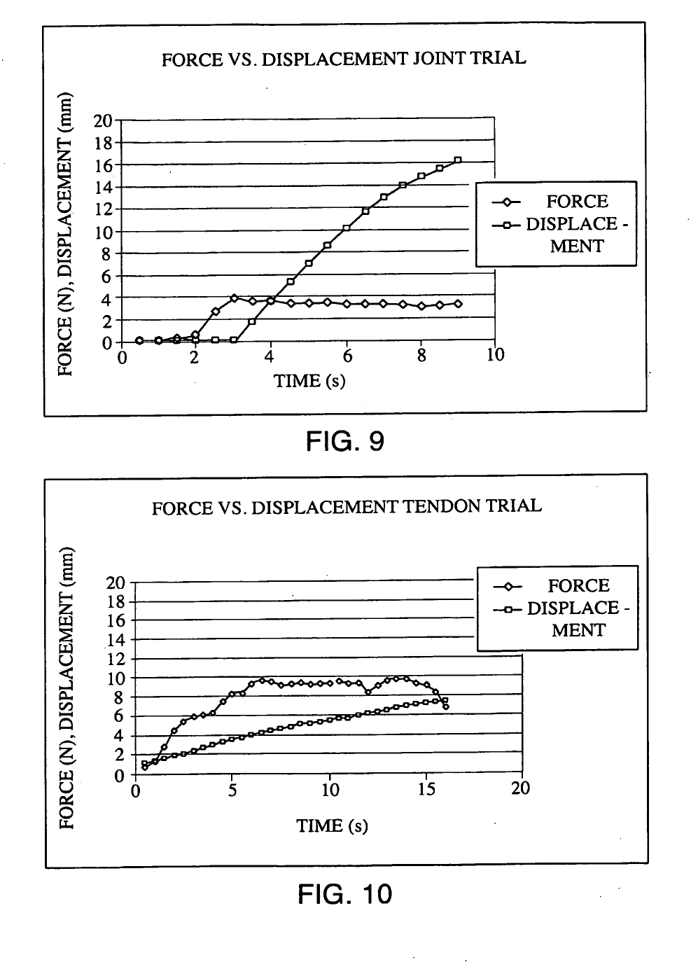

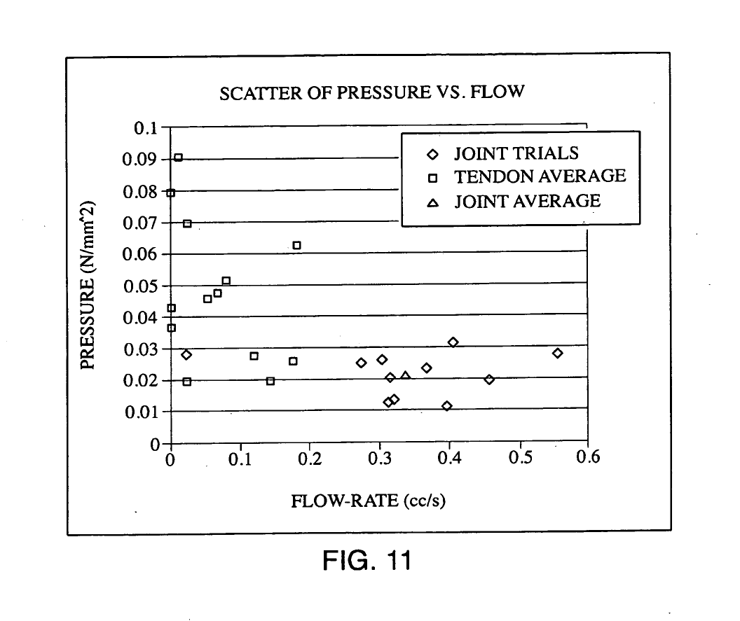

Force and displacement were measured and recorded throughout numerous injections performed in pig tissue on a pig hoof. The data was recorded and used to calculate the pressure and flow-rate during the injections. The maximum pressure during each injection was divided by the average flow-rate of the same injection to calculate an impedance value. As can be seen in FIGS. 9-11 and in Tables 1-2 (below), the fluid flow impedance is generally lower in joint tissue than in other tissue areas. When injecting into an area other than the joint, the average maximum force and pressure were more than twice that of when injecting into the joint. The average flow-rate while injecting into joints was over three times that of tendons. The higher pressure and lower flow-rate of the tendon injections indicates that there is generally higher impedance in these types of tissues compared to joints, which exhibit generally lower pressures and higher flow-rates. When impedance is compared between the two locations, impedance in a tendon is over 550 times that of a joint if all trials are included. Even if the outlying tendon impedance values are omitted, the impedance value of the tendon is over 20 times that of the joint.

TABLE 1
Average Joint Values

| | Max | | | | | |
|---|---|---|---|---|---|---|
| Max Force | Pressure | Displacement | Volume | Flow-Rate | | Impedance |
| (N) | (N/mm\hat{ }2) | (mm) | (cc) | (cc/s) | Time (s) | (N/mm2)/(cc/s) |
| 3.93166667 | 0.02085785 | 17.2375 | 3.04471042 | 0.33735582 | 8.91666667 | 0.06226264 |

TABLE 2
Average Tendon Values

| | Max | | | | | |
|---|---|---|---|---|---|---|
| Max Force | Pressure | Displacement | Volume | Flow-Rate | | Impedance |
| (N) | (N/mm\hat{ }2) | (mm) | (cc) | (cc/s) | Time (s) | (N/mm2)/(cc/s) |
| 8.95633333 | 0.04751478 | 4.88641667 | 0.82311925 | 0.06869617 | 8.54166667 | 34.7925685 |

### Example 2

With reference to FIGS. 1-3, in particular exemplary embodiments, the sensors 30 are a pressure transducer and/or flow sensor that comes into physical contact with the fluid in the syringe body 15b. For example, a pressure and/or flow sensor can be provided in the housing 20 between the needle 25 and the syringe body 15b such that the housing provides a fluid channel that can be generally the same diameter as the fluid channel adjacent the needle such that alteration of the syringe 15 is reduced. Fluid impedance can be tested by a user by inserting the needle 25 into the tissue T1, T2 and T3 and then injecting a relatively small amount of fluid into the tissue T1, T2 and T3. The pressure and flow readings from the sensors 30 can then be converted into impedance, e.g., by the computer device 23 and relayed to the user via the user feedback indicator member 22.

### Example 3

With continued reference to FIGS. 1-3, the sensor(s) 30 are force and/or pressure sensors that measure the impedance or force/pressure on the needle 25 during insertion into the tissue T1, T2, T3. For example, the sensor(s) 30 can be piezoelectric or other suitable force sensors positioned on the needle to detect force as a function of time during needle insertion. In particular embodiments, the force and/or pressure sensor(s) 30 can be in communication with the needle 25 and/or the sensor(s) can detect a deforming force on the needle 25. The force applied to the needle 25 (or on the syringe 15) can be measured while the needle 25 is inserted into the tissue T1, T2, T3. Generally continuous force readings may be taken, and a force curve may be created. For example, the user may apply constant force during insertion, and then the force may drop when the needle 25 reaches the joint cavity tissue T3. The computer device 23 may be configured to detect the reduction in force when the needle 25 reaches the joint cavity tissue T3 and relay the feedback to the user via the user feedback indicator member 22.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

That which is claimed:

1. A syringe system comprising:
a syringe with a syringe body defining a fluid cavity in fluid communication with an injection needle;
a pressure, force and/or flow sensor in fluid communication with the needle; and
a user feedback unit in electrical communication with the sensor and configured to provide user feedback based on data from the pressure, force and/or flow sensor.

2. A syringe system according to claim 1, wherein the user data feedback unit comprises a housing having a light indicator whereby in operation the housing generates a light if the needle is in a suitable location for injection.

3. A syringe system according to claim 2, wherein the housing is configured to releasably engage the syringe body.

4. A syringe system according to claim 3, wherein the housing is configured to slidably snugly receive a portion of the syringe therein.

5. A syringe system according to claim 2, wherein the housing comprises a digital signal processor circuit configured to calculate an index of resistance associated with the location of the needle in a patient with a low index of resistance indicating a desired injection site.

6. A syringe system according to claim 5, further comprising a portable computer device in communication with the sensor and the housing to programmatically direct the output of the user feedback unit.

7. A syringe system according to claim 5, wherein the sensor is configured to wirelessly communicate with the digital signal processor circuit.

8. A syringe system according to claim 6, wherein the sensor and the user feedback unit are configured to wirelessly communicate with the digital signal processor circuit.

9. A syringe system according to claim 2, wherein the housing is serially reusable with different syringes and a respective syringe is single-use disposable.

10. A syringe system according to claim 1, wherein the syringe is a teaching syringe for teaching a user to associate a tactile excursion feel associated with a low index of resistance.

11. A syringe system according to claim 1, wherein the syringe is configured and sized as an orthopedic tool to deliver medicament accurately to a target joint space of an arthritic patient.

12. A syringe system according to claim 1, wherein the syringe comprises a plunger, and the pressure and/or flow sensor comprises a plunger force sensor configured to detect a force exerted on the plunger by a user and a displacement sensor configured to detect a displacement of the plunger.

13. A syringe system according to claim 12, wherein the user feedback unit comprises a digital signal processor circuit configured to calculate an index of resistance based on the force exerted on the plunger and the displacement of the plunger with a low index of resistance indicating a desired injection site.

14. A syringe system according to claim 13, wherein the index of resistance is based on a test injection.

15. A syringe system according to claim 12, wherein the user data feedback unit comprises a housing configured to releasably attach to the plunger of the syringe and to having the plunger force sensor and displacement sensor thereon.

16. A syringe system according to claim 1, wherein the pressure and/or flow sensor comprises a fluid pressure sensor and a flow meter.

17. A syringe system according to claim 1, wherein the force, pressure and/or flow sensor comprises a force sensor configured to detect a force on a portion of the syringe as a user inserts the injection needle into tissue of a patient.

18. A syringe system according to claim 1, wherein the user feedback unit is attached to the syringe body.

19. A syringe system comprising:
a syringe with a syringe body defining a fluid cavity and having a plunger in the fluid cavity, the fluid cavity being in fluid communication with an injection needle;
a plunger force sensor configured to detect a force exerted on the plunger by a user;
a displacement sensor configured to detect a displacement of the plunger; and
a user feedback unit in electrical communication with the plunger force sensor and the displacement sensor and configured to provide user feedback based on data from the pressure and/or flow sensor.

20. A syringe system according to claim 19, wherein the user data feedback unit comprises a housing having a light indicator whereby in operation the housing generates a light if the needle is in a suitable location for injection.

21. A syringe system according to claim 20, wherein the housing is configured to releasably engage the syringe body.

22. A syringe system according to claim 21, wherein the housing is configured to slidably snugly receive a portion of the syringe therein.

23. A syringe system according to claim 20, wherein the housing comprises a digital signal processor circuit configured to calculate an index of resistance associated with the location of the needle in a patient with a low index of resistance indicating a desired injection site.

24. A syringe system according to claim 23, further comprising a portable computer device in communication with the sensor and the housing to programmatically direct the output of the user feedback unit.

25. A syringe system according to claim 23, wherein the sensor is configured to wirelessly communicate with the digital signal processor circuit.

26. A syringe system according to claim 24, wherein the sensor and the user feedback unit are configured to wirelessly communicate with the digital signal processor circuit.

27. A syringe system according to claim 21, wherein the housing is serially reusable with different syringes and a respective syringe is single-use disposable.

28. A syringe system according to claim 20, wherein the syringe is a teaching syringe for teaching a user to associate a tactile excursion feel associated with a low index of resistance.

29. A syringe system according to claim 20, wherein the syringe is configured and sized as an orthopedic tool to deliver medicament accurately to a target joint space of an arthritic patient.

30. A syringe system according to claim 20, wherein the user feedback unit comprises a digital signal processor circuit configured to calculate an index of resistance based on the force exerted on the plunger and the displacement of the plunger with a low index of resistance indicating a desired injection site.

31. A syringe system according to claim 29, wherein the index of resistance is based on a test injection.

32. A syringe system according to claim 30, wherein the user data feedback unit comprises a housing configured to releasably attach to the plunger of the syringe and to having the plunger force sensor and displacement sensor thereon.

33. A syringe system according to claim 1, wherein the user feedback unit is attached to the syringe body.

34. A syringe sensor system configured to releasably attach to a syringe with a syringe body defining a fluid cavity and having a plunger in the fluid cavity, the fluid cavity being in fluid communication with an injection needle, the syringe sensor system comprising:
at least one housing configured to releasably attach to the syringe;
a plunger force sensor on the at least one housing and configured to detect a force exerted on the plunger by a user;
a displacement sensor on the at least one housing and configured to detect a displacement of the plunger; and
a user feedback unit in electrical communication with the plunger force sensor and the displacement sensor and configured to provide user feedback based on data from the force and/or flow sensor.

35. A method for selecting a tissue region for injection with a syringe in communication with an injection needle, the method comprising:
detecting a force, pressure and/or flow associated with the syringe and/or injection needle during insertion of the needle and/or during injection of a fluid through the needle; and
providing user feedback responsive to the detected force, pressure and/or flow.
